(19) United States
(12) Patent Application Publication
(10) Pub. No.: US 2007/0144537 A1
(43) Pub. Date: Jun. 28, 2007

(54) INSTRUMENT GUIDE AND METHOD FOR USE

(76) Inventors: Salvatore Privitera, Mason, OH (US); Jeffery A. Gonya, Dayton, OH (US)

Correspondence Address: FROST BROWN TODD LLC, 2200 PNC Center, 201 East Fifth Street, Cincinnati, OH, 45202, US

(21) Appl. No.: 11/609,068

(22) Filed: Dec. 11, 2006

Related U.S. Application Data
(63) Continuation-in-part of application No. 11/028,901, filed on Jan. 3, 2005.

(51) Int. Cl. A61B 19/08

(52) U.S. Cl. 128/852

(57) ABSTRACT

A guide for a surgical instrument is disclosed. The instrument guide has an elongate bifurcated flexible body having a first flexible member and a second flexible member joined together at a fork. The fork is located at a distal end of the bifurcated flexible body and the first flexible member has a first free end and the second flexible member has a second free end. A pocket is attached to the elongate bifurcated flexible body about the fork. An anchor positioned on or near to one of the first free end or the second free end, the anchor attaching the guide to a surgical instrument. And, a connector can be provided to attach the guide to a surgical instrument. The connector comprises a first portion of the connector on the surgical instrument and a second portion of the connector about a first end of the guide. When the second portion of the connector is operably connected with the first portion of the connector, at least a portion of the connector rotates.

### CROSS REFERENCE TO RELATED APPLICATIONS

This is a continuation-in-part of application Ser. No. 11/028,901 filed on Jan. 13, 2005.

### BACKGROUND OF THE INVENTION

The present invention relates to surgical instruments, with one embodiment relating to guides for surgical instruments. Surgery generally refers to the diagnosis or treatment of injury, deformity, or disease. Most surgical procedures involve the use of one or more surgical instruments. In most cases, the surgical instrument must be positioned on or near target tissue of the patient. Target tissue refers to the desired destination, whether final or intermediate, of a surgical instrument, and may or may not include the tissue that is the subject of surgery. A variety of factors may make the placement of the surgical instrument challenging. For example, the shape, position, or sensitivity of the target tissue or surrounding anatomy may complicate positioning of a surgical instrument. As a further example, the geometry of the surgical instrument may pose complications in positioning the instrument. In yet another example, the nature or timing of the procedure may complicate placement of the surgical procedure. Another example is the size of the surgical field. Still other examples of circumstances may complicate positioning of surgical instruments. Instrument guides are sometimes used to facilitate placement of surgical instruments. No one, however, has previously made or used an instrument guide in accordance with the present invention.

### BRIEF DESCRIPTION OF THE FIGURES

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 illustrates an oblique view of an instrument guide;

FIG. 2 illustrates an oblique view of a bobbin for holding an instrument guide;

FIG. 3 illustrates an instrument guide with a positioning mechanism;

FIG. 4 illustrates an instrument guide attached to a surgical instrument;

FIG. 5 illustrates another example of an instrument guide;

FIG. 6 illustrates an instrument guide attached to a surgical instrument;

FIG. 7 illustrates another example of an instrument guide having a bifurcated elongate flexible body;

FIG. 8 illustrates the instrument guide of FIG. 7 attached to a surgical instrument.

FIG. 9 illustrates an example of an anchor used to attach an instrument guide to a surgical instrument; and

FIG. 10 illustrates the anchor of FIG. 9 releasably attached to the surgical instrument.

### DETAILED DESCRIPTION OF THE INVENTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive

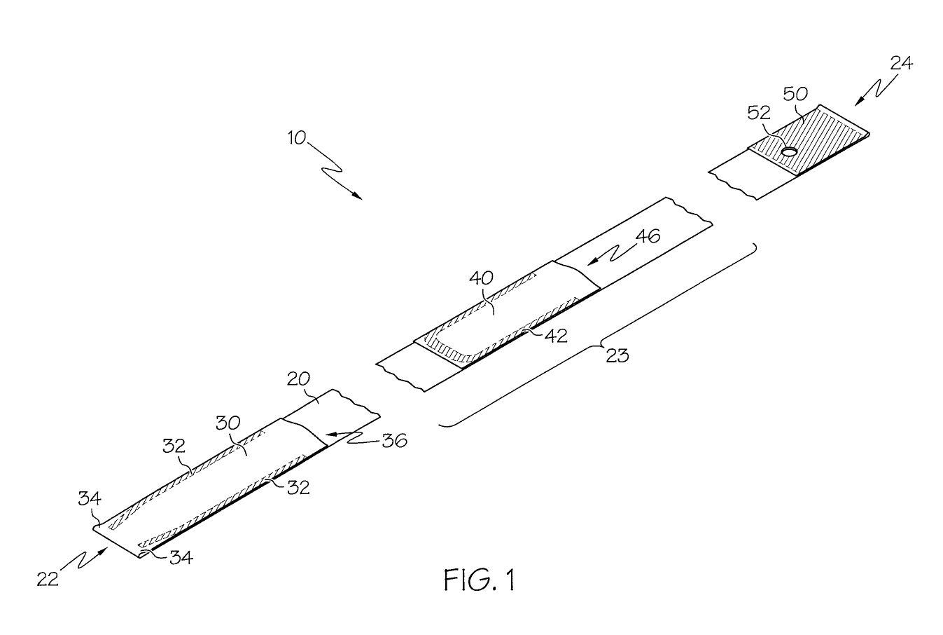

FIG. 1 illustrates an example of an instrument guide (10). The instrument guide (10) includes an elongate flexible strip (20) having a distal end (22), a proximal end (24), and a medial portion (23) between the proximal and distal ends. Optionally, the elongate flexible strip (20) is formed at least in part of an elastomer. One suitable material is a polyurethane, such as PELETHANE series 2363 by DOW PLASTICS, but other materials may also be used. In the present embodiment, the elongate flexible strip (20) is in the form of a ribbon; however, other geometries could also be employed. The dimensions of the elongate flexible strip (20) may vary substantially, and in this example the ratio of width to length is about 0.01 to about 0.03. In the present embodiment, the elongate flexible strip (20) is about 30 to about 40 inches long, about 0.4 to about 0.8 inches wide, and about 0.001 to about 0.005 inches thick. For example, one embodiment has a nominal length of about 36 inches, a nominal width of about 0.75 inches, and a nominal thickness of about 0.003 inches.

A first pocket (30) is positioned adjacent the distal end (22) of the elongate flexible strip (20). As shown here, the distal pocket (30) has an opening (36) on the proximal end of the pocket, and the distal end of the pocket is substantially closed. The distal pocket (30) is formed by folding the elongate flexible strip (20) onto itself and adhering the walls to form seals (32). Heat or RI sealing or use of an adhesive are two exemplary techniques to adhere the walls. As shown here, the seals (32) tapered toward the distal end of the pocket. Two tabs (34) are provided at the distal end (22). Optionally, at least a portion of the distal pocket (30) is translucent or transparent.

The present embodiment has a second pocket (40) positioned proximally to the distal pocket (30) on the elongate flexible strip (20). As shown here, the second pocket (40) is positioned on the medial portion (23) of the elongate flexible strip (20). In this embodiment, the medial pocket (40) has an opening (46) on the proximal end of the pocket, and the distal end of the pocket is completely closed. The medial pocket (40) is formed by placing a section of material over the elongate flexible strip (20), preferably but not necessarily the same material, and adhering the walls to form the seal (42). As shown in this example, the distance from the distal end (22) of the elongate flexible strip (10) to the distal end of the medial pocket (40) is between about 15 inches to about 30 inches. In one embodiment, the nominal distance is about 21 inches.

An anchor (50) is positioned adjacent the proximal end (24) of the elongate flexible strip (20). The anchor (50) of the present embodiment includes a hole or eyelet (52), but other attachment mechanisms may be employed, such as holes, slits, hooks, fasteners, etc.

Further, the anchor may have more than one anchor position. As shown here, the anchor (50) is formed by folding the elongate flexible strip (20) onto itself and adhering the walls together, such as with a heat seal or adhesive, thus increasing the wall thickness of the anchor (50). One advantage of increased wall thickness is to reinforce the eyelet (52). As shown in this example, the distance from the distal end of the medial pocket (40) to the eyelet (52) is between about 12 inches to about 13 inches. One embodiment has a nominal distance of about 12.5 inches.

One exemplary use of the instrument guide (10) is to deploy a surgical instrument. A positioning mechanism is placed in the distal pocket (30), and then by using the positioning mechanism the distal pocket (30) is advanced adjacent target tissue. The distal pocket (30) is preferably dimensioned to receive the positioning mechanism. The type positioning mechanism may vary widely, depending, for example, on the geometry of the target tissue and the surrounding anatomy. For instance, the positioning mechanism could be a surgeon's finger, a dissector, forceps, an articulated probe, and the like. The positioning mechanism may then be removed from the distal pocket (30). The tabs (34) may facilitate the removal of the positioning mechanism by providing a surface that the surgeon may grasp, such as with their fingers or with forceps, and pull the distal pocket (30) from the positioning mechanism.

The surgical instrument is placed in the medial pocket (40). The surgical instrument will vary depending upon the treatment and procedure being conducted in the surgery. The medial pocket (40) is preferably dimensioned to receive the appropriate surgical instrument. The elongate flexible strip (10) is then advanced further, such as by pulling the distal end (22) until the surgical instrument is adjacent the target tissue. The surgical instrument is removed from the medial pocket (40), such as by continuing to advance the elongate flexible strip (10) while keeping the surgical instrument stationary. Thus, the surgical instrument is successfully positioned adjacent the target tissue. The remainder of the elongate flexible strip (10) may then be removed from the surgical field. Optionally, the elongate flexible strip (10) may remain in the surgical field, before or after the surgical instrument is deployed, and function as a sling to hold or position tissue. Preferably, the portions of the instrument guide (10) that contact tissue in the surgical field may be free of sharp or abrasive edges.

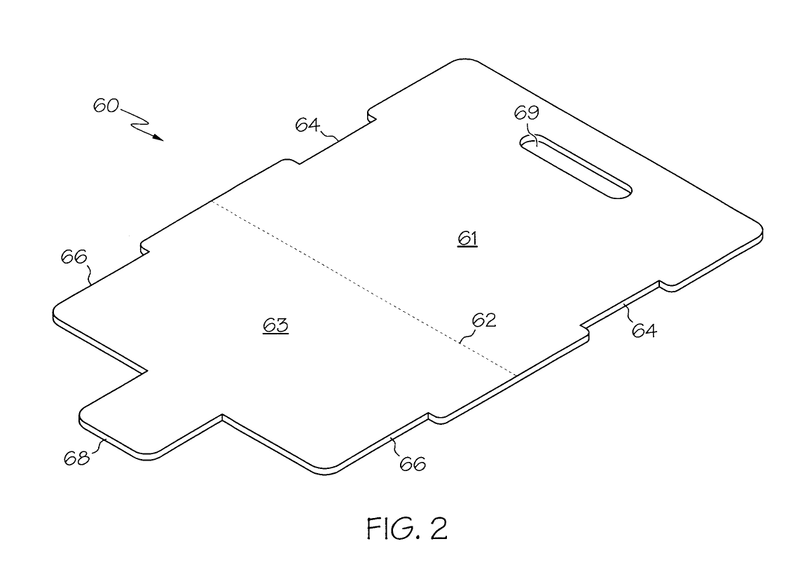

FIG. 2 illustrates an optional bobbin (60) for holding the instrument guide (10). The bobbin (60) comprises a sheet of material foldable along a crease (62) that separates a base portion (61) and a cover portion (63). The bobbin (60) has an opened position (as shown in the figure) and a closed position where the cover portion (63) is folded onto the base portion (63). In the closed position, the recesses (64, 66) align with one another. The tab (68) mates with the slot (69) to function as a fastener to selectively hold the bobbin (60) in the closed position. Naturally, alternative fastening mechanisms may also be used. As shown in this embodiment, the bobbin (60) is made from a single sheet of material, such as 0.01 inch LEXAN 8040. The sheet has a nominal width of about 3 inches and a nominal length of about 5 inches.

In the opened position, the instrument guide (10) is wrapped between the recesses (64) starting the with proximal end (24). Before the entirety of the elongate flexible strip (20) is wrapped, the cover portion (63) is closed and fastened, and the remainder of the instrument guide (10) is wrapped between the recesses (66). The medial pocket (40) may be wrapped between recesses (64) in the opened position; however, the medial pocket could also be wrapped between recesses (66) in the closed position. The completely wrapped instrument guide (10) and bobbin (60) may then be stored in a sterilized and sealed pouch with instructions. Once the pouch opened, the surgeon unwraps a portion instrument guide (10) without releasing the tab (68). Thus, the surgeon may work with the distal pocket (30) and a portion of the elongate flexible strip (20) while the remainder of the instrument guide (10) is contained in the closed bobbin (60) and out of the way during the surgery. Once the surgeon is ready to advance the surgical instrument into the surgical field, the bobbin (60) may be opened and the remainder of the instrument guide (10) unwrapped and used. The bobbin (60) may then be discarded.

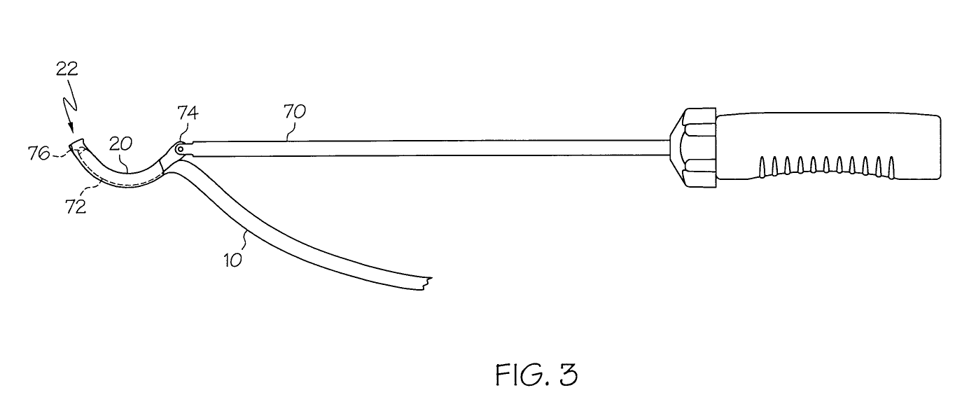

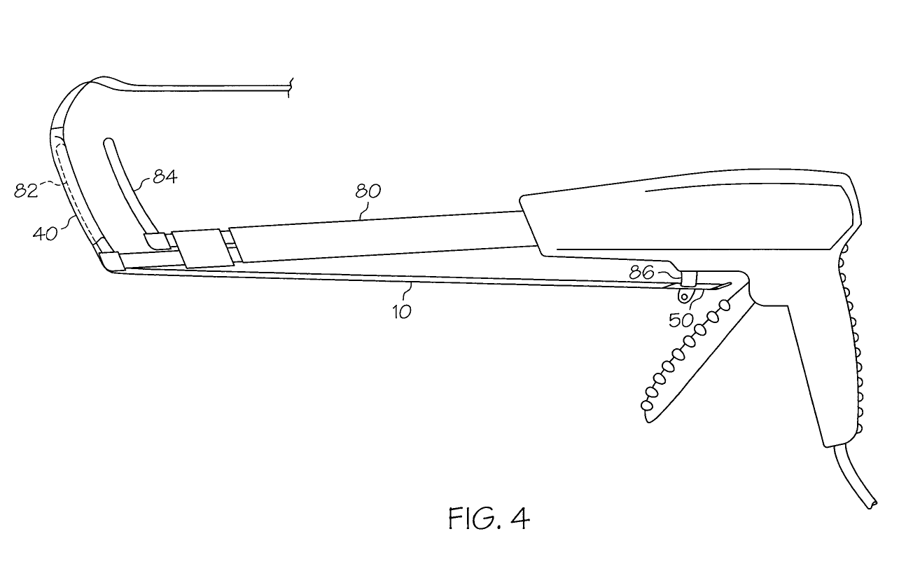

FIGS. 3 and 4 illustrate an example of a procedure to use the instrument guide (10) in conjunction with a treatment for atrial fibrillation. It should be appreciated that numerous other surgical procedures, anatomies, and surgical instruments may be used, and the present example is merely an illustration of one embodiment of the invention. The positioning mechanism (70) in this example, shown here as a dissector, is placed in the distal pocket (30). The positioning mechanism (70) has an articulated shaft and includes an arcuate segment (72) that pivots about the joint (74). The arcuate segment (72) has blunt and rounded distal end (76). A light source emits visible energy from the distal end (76) that facilitates, among other things, locating the distal end (76) during a procedure and differentiating tissue. In this example, the distal pocket (30) is translucent.

The following describes an exemplary procedure using the positioning mechanism (70) to separate the left or right pair of pulmonary veins adjacent the left atrium. The procedure may be performed during open or minimally invasive surgery. With the arcuate segment (72) in the distal pocket (30), the distal end (76) of the arcuate segment (72) is positioned adjacent the junction of one of the pulmonary veins (superior or inferior) and the left atrium. The distal end (76) is advanced around the posterior of the pair of pulmonary veins while simultaneously pivoting the arcuate segment (72). The distal end (76) continues to advance until it emerges beyond the other adjacent pulmonary vein (the inferior or superior, as the case may be). The advancement of the distal end (76) separates the pair of pulmonary veins from the pericardial reflections, thus creating a dissected path between the pulmonary veins and the pericardium. The dissected path may be widened by sweeping the arcuate segment (72) and further separating the tissue and widening the dissected path. The surgeon may grasp the distal end (22) of the instrument guide (10) and pull the distal pocket (30) from the arcuate segment (72). The arcuate segment (72) may then be backed out and removed from the surgical field.

As illustrated in FIG. 4, the surgical instrument (80) in this example is a surgical clamp having a distal jaw (82) and a proximal jaw (84). In this example, the surgical instrument (80) is used to ablate tissue with RF energy (one example is disclosed in U.S. Pat. No. 6,517,536). One of the jaws, in this example the distal jaw (82), is placed in the medial pocket (40) of the instrument guide (10). The anchor (50) attaches to surgical instrument (80) to prevent the distal jaw (82) from inadvertently liberating from the medial pocket (40). As shown in this example, the anchor (50) attaches to the clamp release lever (86) and the instrument guide (10) remains taut between the anchor (50) and medial pocket (40). By continuing to pull the distal end (22) of the instrument guide (10), the distal jaw (82) may be accurately positioned in the dissected path until the pulmonary veins are interposed between the distal and proximal jaws (82, 84). The anchor (50) may then be detached. By pulling instrument guide (10), the medial pocket (40) may be pulled from the distal jaw (82). The instrument guide (10) may be removed from the surgical field. Alternatively, the instrument guide (10) can remain in the surgical field to hold or lift the vessels like a sling. The distal and proximal jaws (82, 84) may then be positioned such that the tissue being treated is interposed between the jaws. In one example, the tissue being treated is the atrium wall adjacent the pulmonary veins. The jaws may then be closed and the tissue ablated.

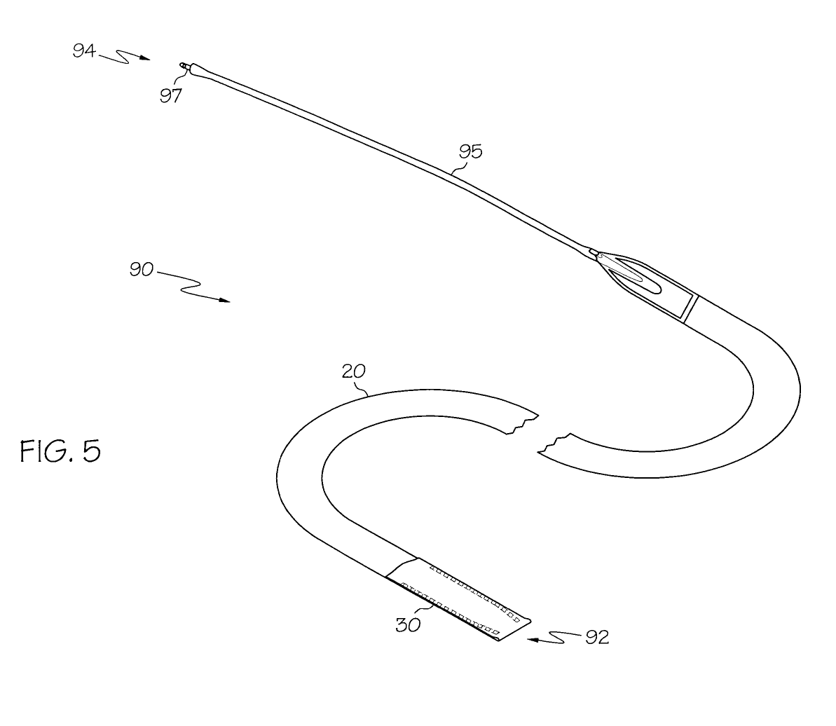

FIG. 5 illustrates another example of an instrument guide (90). The instrument guide (90) has an elongate flexible body with a distal end (92) and a proximal end (94). In the present example the body comprises two or more portions. While geometry or and materials in the portions can vary, as shown here the distal portion is an elongate flexible strip (20) similar to instrument guide (10), and the proximal portion is an elongate flexible member (95) having a generally round cross-sectional geometry. While both portions are flexible, the proximal portion is more resilient than the distal portion. In the present example the flexible member (95) has a generally round cross-sectional geometry (solid or tubular) and is formed from a flexible resilient material (such as rubber or durometer santoprene). The dimensions of the two portions can vary, but in one embodiment, the proximal portion (95) is between about 6 and about 14 inches in length, and the distal portion (20) is between about 15 and about 20 inches in length.

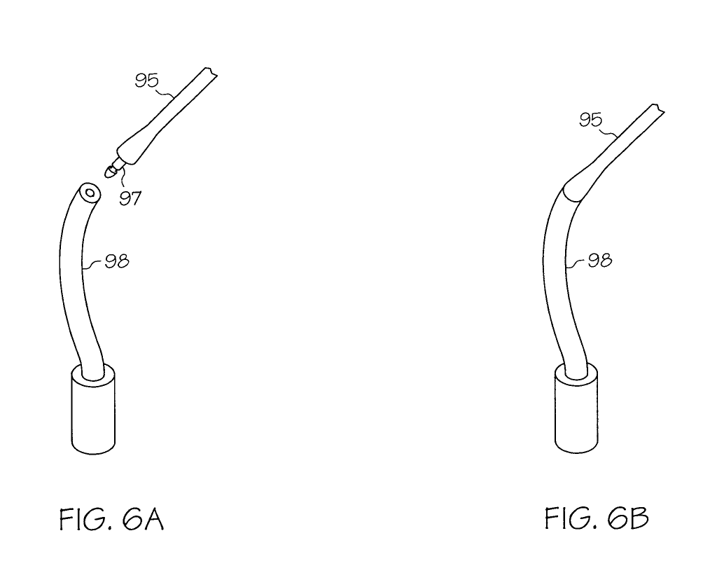

Located on or near the proximal end (94) is an anchor (97) adapted for attaching the guide (90) to a surgical instrument. As shown in FIGS. 6A&B, the anchor (97) is a male prong fastener dimensioned to engage a female fastener counterpart located on the surgical instrument (98). The guide (90) can be separated from the surgical instrument by pulling the anchor (97) in the distal direction. Preferably, a threshold pulling force is required to prevent inadvertent separation. Thus, the anchor (97) can be selectively engaged and disengaged from the surgical instrument. Alternatively, the anchor may be permanently attached to the surgical instrument. Naturally, the anchor (97) can take a variety of other forms other than the male/female arrangement described in this example, including without limitation screws or threads, snaps, adhesives, interference fits, barbs, magnets, and the like. In the present example, the surgical instrument (98) is the distal jaw of an ablation clamp; however, a variety of other surgical instruments could also be used depending on the surgical procedure and technique. As shown here, the guide (90) extends axially in-line with the jaw; however, the guide (90) could also extend at an angle from the jaw axis. Alternatively, the anchor (97) could pivotally attach to the jaw, such as with a ball and socket joint.

The following illustrates one exemplary method for using the instrument guide (90). Similar to the example illustrated in FIG. 3, a positioning mechanism is placed in the distal pocket (30). A dissection path is created between the pulmonary veins and the pericardium using the positioning mechanism. The surgeon may grasp the distal end (92) of the instrument guide (90) and pull the distal pocket (30) from the positioning mechanism. The positioning mechanism may then be backed out and removed from the surgical field. The anchor (97) is attached to the jaw (98), either before or after the dissected path is created. By continuing to pull the distal end (92) of the instrument guide (90), the distal jaw (98) may be accurately positioned in the dissected path until the pulmonary veins are interposed between the distal and proximal jaws of the ablation clamp. With the anchor (97) attached to the jaw (98), the distal and proximal jaws may then be positioned such that the tissue being treated is interposed between the jaws. The jaws may then be closed and the tissue ablated. After treatment is concluded, the distal jaw (98) is backed out of the dissection path thus pulling the instrument guide (90) behind the jaw (98) until the instrument guide (90) is removed from the surgical field.

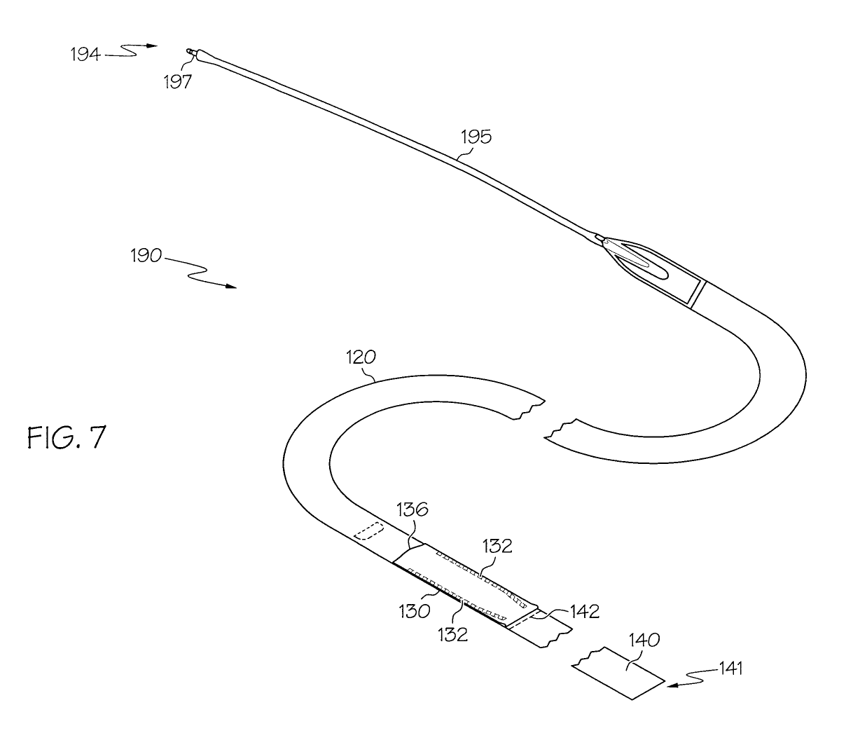

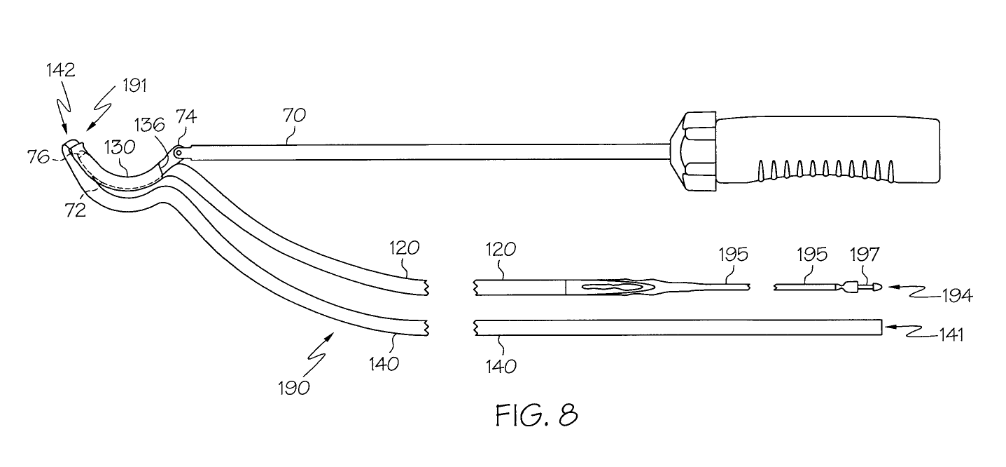

FIG. 7 illustrates yet another alternate embodiment of a bifurcated instrument guide (190) of the present disclosure. The bifurcated instrument guide (190) of the present example has a flexible body comprising a first flexible member (120) and a second flexible member (140) connected together at a fork or first end (191) (FIG. 8). The first flexible member (120) and the second flexible member (140) extend proximally from the first end (191) and may be formed from one piece, or can be separate pieces that are joined connected, adhered, or fastened together by seals (132), adhesives, heat staking, fasteners and the like. The first flexible member (120) and the second flexible member (140) are shown in an extended flat condition in FIG. 7 but a fold (142) can be placed at the joining or in one member such as flexible member (140) and can be about the connection of the first flexible member (120) to the second flexible member (140). This fold (142) creates the fork of the bifurcated instrument guide (190) as shown in FIG. 8. The fold (142) also forms the distal or first end (191) of the bifurcated instrument guide (190) when the first flexible member (120) and the second flexible member (140) are folded parallel to one another.

The second flexible member (140) can be formed from an elongate flexible strip described previously as instrument guide (10), and can be similar in size, shape, and materials. The first flexible member (120) may differ from the second flexible member (140) and can have both a first distal portion and a second proximal portion. The first distal portion can comprise an elongate flexible strip and the second proximal portion can comprise a cylindrical proximal portion or elongate flexible member (195). The flexible member (195) may have a generally round cross-section and can be solid or hollow. Flexible member can be formed from a flexible resilient or elastomeric material such as rubber or santoprene.

The bifurcated instrument guide (190) has the distal or first end (191), and first flexible member (120) can terminate at a first free end (194) and second flexible member can terminate at a second free end (141). An anchor (197) can be located at the first free end (194) of first flexible member (120).

A first pocket (130) may be provided at or near to the fork or first end (191) and/or fold (142) of the instrument guide (190), and can have an opening (136) for the reception of a surgical instrument such as a positioning mechanism (70) therein. First pocket (130) can be attached to the first flexible member (120) with the same joining used to attach the second flexible member (140), or can be attached with separate joinings. As shown, the seals (132) join both the pocket (130) and the second flexible member (140) to the first flexible member (120).

As shown in FIG. 8, the elongate flexible strip portion of the first flexible member (120) and the second flexible member (140) can have about the same length. Alternately by way of examples, the length of the second flexible member (140) can be less than the first flexible member (120), about the same as the length as the sum of the first portion and the second portion of the first flexible member (120), or longer than the first flexible member (120). Second flexible member (140) can vary in length between about 1 inch and about 35 inches in length.

The following illustrates an example of a method for using the bifurcated instrument guide (190). As shown in FIG. 8, a distal end of a positioning mechanism (70) is placed in an opening (136) of the distal pocket (130), and the free ends (194), (141) of the first flexible member (120) and the second flexible member (140) respectively, are drawn together. Next, the positioning mechanism (70) and attached bifurcated instrument guide (190) are used to create a dissection path between the pulmonary veins and the pericardium and to expose the first end (191) of the bifurcated instrument guide (190) to the surgeon. Once the dissection path is created, the surgeon has two options to use the instrument guide (190).

With the first option, the surgeon may grasp the first end (191) of the bifurcated flexible instrument guide (190) and pull the distal pocket (130) from the positioning mechanism. The positioning mechanism is removed from the surgical site. The anchor (197) of the bifurcated guide (190) may be attached to the distal jaw (98) of the previously described surgical instrument such as an ablation clamp, either before or after the dissected path is created. Once the bifurcated guide is attached to the distal jaw (98) of the ablation clamp, pulling on the first end (191) of the bifurcated instrument guide (190), can draw or guide the distal jaw (98) until the ablation clamp is accurately positioned in the dissected path with the pulmonary veins interposed between the distal and proximal jaws. The jaws may then be closed and the tissue ablated. After treatment is concluded, the distal jaw (98) is backed out of the dissection path thus pulling the instrument guide (190) behind the jaw (98) until the instrument guide (90) is removed from the surgical field.

With the second option, the surgeon can reach in and grasp the second flexible member (140) proximal to the fork or fold (142) and pull on the second flexible member (140) until the second free end (141) is withdrawn from the patient. With the second flexible member (140) deployed in this manner, the positioning mechanism (70) may then be backed out and removed from the surgical field and the bifurcated instrument guide (190) can remain in the surgical field. The bifurcated instrument guide (190) can function as a sling to hold or position tissue and/or can be used as an aid to place the surgical instrument into position.

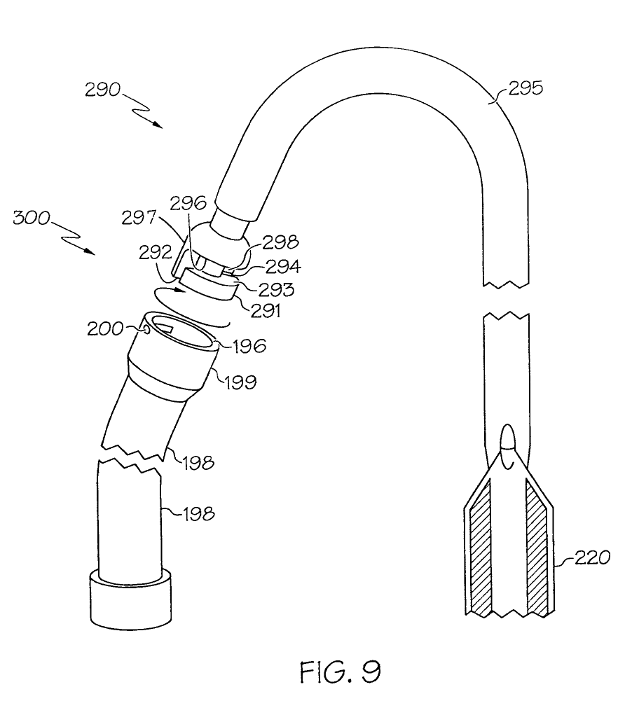

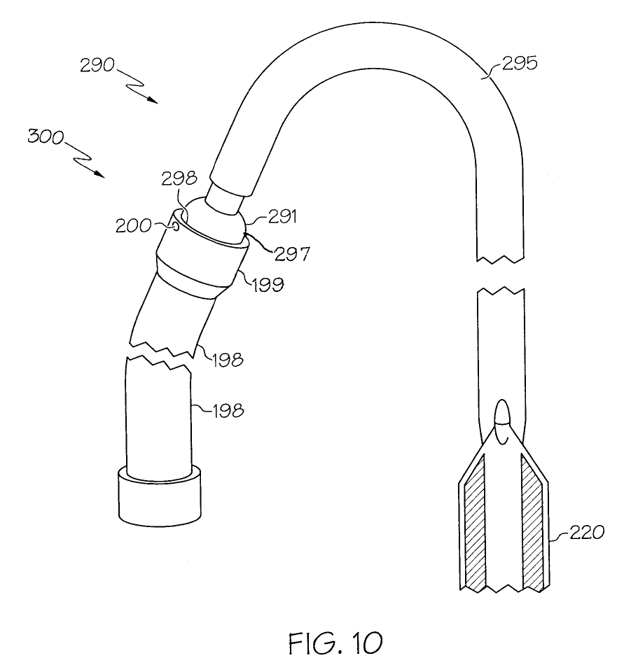

FIGS. 9-10 show an alternate embodiment of a connector (300) that can removably connect or attach a guide (290) to an instrument jaw (198) of a surgical instrument. The connector (300) comprises an anchor (297) at a distal end of the guide (290) and an anchor receiver (199) at a distal end of the instrument jaw (198). The anchor receiver (199) comprises a socket (196) and one or more male members or pins (200) extending into socket (196). The anchor (297) comprises a rigid cylinder (291) that extends from an elastomeric flexible member (295) and can be linearly received in socket (196). The cylinder (291) has at least one female member or slot (292) extending therein to allow passage or linear movement of pin (200) into slot (292) as cylinder (291) is linearly received in socket (196). When the anchor (297) is fully placed linearly into socket (196), an elastomeric edge (296) of flexible member (295) is slightly compressed as pin (200) linearly aligns with a second female member or radial slot (293) in cylinder (291). When pin (200) is aligned with radial slot (293), the anchor (297) can be rotated clockwise to traverse pin (200) about radial slot (293). As shown, radial slot (293) allows between about 5 degrees and about 359 degrees of rotational motion, before pin (200) contacts a stop (298). As shown in FIG. 9, when anchor (297) is released by the operator at stop (298), the elastomeric edge (296) un-compresses and lifts the anchor (297) to engage pin (200) in a detent slot (294) adjacent to the stop (298). Detent slot (294) provides a rotary detent to prevent rotation of anchor (297), and ensures that the anchor (297) remains connected with anchor receiver (199). Counter-clockwise rotation of anchor (297) can overcome the detent action of pin (200) in detent slot (294), and after full rotation of pin (200) in radial slot (293), pin (200) is moved back into the axial slot (292). Further axial motion of anchor (297) can disconnect the connector (300) and release the guide (290) from the instrument jaw 198. Guide (290) is shown as a flexible member (295), the anchor (297) at a proximal end, and a flexible member (220) attached at distal end, the flexible member (220). However, guide (290) is not limited to the guide shown, but by way of example can be and is not limited to any guide member described previously such as a ribbon or a bifurcated ribbon.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

What is claimed is:

1. A guide for a surgical instrument, comprising:
a) an elongate bifurcated body comprising a fork with a first flexible member and a second flexible member extending therefrom, the first flexible member having a first free end and the second flexible member having a second free end;
b) a pocket attached to the elongate bifurcated body about the fork; and
c) an anchor positioned on or near to one of the first free end or the second free end, the anchor for attaching the guide to a surgical instrument.

2. The guide of claim 1, wherein at least a portion of the elongate bifurcated body is an elastomeric ribbon.

3. The guide of claim 1, wherein at least a portion of the elongate bifurcated body is translucent or transparent.

4. The guide of claim 1, wherein the first flexible member and the second flexible member are separate pieces joined together.

5. The guide of claim 1, wherein the fork is a fold to bring the first flexible member about parallel to the second flexible member to form the elongate bifurcated body.

6. The guide of claim 1, wherein the first flexible member and the second flexible member are about the same length.

7. The guide of claim 1, wherein the one of the first flexible member or the second flexible member having the anchor has at least a portion of a generally round cross-sectional geometry between the anchor and the fork.

8. The guide of claim 1, wherein the pocket is attached to at least one of the first flexible member and the second flexible member.

9. The guide of claim 1, wherein the pocket and the anchor are attached to the same one of the first flexible member and the second flexible member.

10. The guide of claim 1, wherein the anchor has at least one member to engage with the surgical instrument and when the anchor attaches to a surgical instrument, at least a portion of the member moves linearly to attach to the surgical instrument.

11. The guide of claim 1, wherein the anchor has at least one member to engage with the surgical instrument and when the anchor attaches to a surgical instrument, at least a portion of the feature rotates to attach to the surgical instrument.

12. A method for using a bifurcated guide in a patient, the method comprising:
a) attaching a fork of a bifurcated guide to a positioning mechanism, the bifurcated guide having a fork at a distal end and a first flexible member and a second flexible member extending proximally threrefrom;
b) advancing the bifurcated guide about selected tissue with the positioning mechanism until the fork is exposed; and
c) selecting a portion of the bifurcated guide to pull, the portion selected from the group consisting of: the fork, the first flexible member, and the second flexible member.

13. The method of claim 12 wherein the fork is selected and further including a step of releasing the bifurcated guide from the positioning instrument.

14. The method of claim 13 wherein the bifurcated guide further comprises a surgical instrument attached to one proximal end of the first flexible member or the second flexible member and including a step of drawing the fork of the bifurcated guide from the patient to draw the surgical instrument into position about the selected tissue.

15. The method of claim 14 further including a step of treating the patient with the surgical instrument.

16. The method of claim 12 wherein one of the first flexible member or the second flexible member is selected and further including a step of drawing the selected one of the first flexible member or the second flexible member from the patient to create a sling about the selected surgical site.

17. The method of claim 16 further including a step of positioning tissue at the selected surgical site with the sling.

18. The method of claim 16 wherein the bifurcated guide further comprises a surgical instrument attached to a free end of the first flexible member or the second flexible member and including a step of drawing the sling from the patient to draw the surgical instrument into position about the selected tissue.

19. A connector for attaching a guide to a surgical instrument, comprising:
a) a first portion of the connector on the surgical instrument; and
b) a second portion of the connector about a first end of the guide, wherein when the second portion of the connector is attached to the first portion of the connector, at least a portion of the connector rotates.

20. The connector of claim 19, wherein at least a portion of the connector moves linearly during connection of the guide to the surgical instrument.

21. The connector of claim 20, wherein the connector further comprises at least one detent.

22. The connector of claim 19, wherein the connector connects the guide to the surgical instrument by engaging an at least one male member with an at least one female member.

23. The connector of claim 22, wherein the female member is at least one slot and the male member is at least one pin.

24. The connector of claim 23, wherein the first portion of the connector of the surgical instrument further comprises the at least one pin.

25. The connector of claim 24, wherein the second portion of the connector of the guide further comprises the at least one slot engagable with at least one pin.

26. The connector of claim 23, wherein the at least one slot has a linear portion for linear movement of the pin therein.

27. The connector of claim 23, wherein the at least one slot has a rotational portion for rotational movement of the pin therein.

28. A method for removably attaching a guide to a surgical instrument, comprising
a) ringing the guide into contact with the surgical instrument; and
b) rotating at least a portion of the guide or the surgical instrument to attach the guide to the surgical instrument.

29. The method of claim 28 further including a step of moving the guide or the surgical instrument linearly when attaching the guide to the surgical instrument.

30. The method of claim 28 wherein the guide and the surgical instrument further comprise a male member and a female member and further including the step of engaging the male member with the female member when attaching the guide to the surgical instrument.
